(19) United States
(12) Plant Patent Application Publication
(10) Pub. No.: US 2020/0060055 P1
(43) Pub. Date: Feb. 20, 2020

(54) MEDINILLA PLANT NAMED 'ROYAL TWIST'

(50) ;

(71) Applicants: Elly Bak, Rijsenhout (NL); Nicolaas D.M. Steur, Oude Niedorp (NL)

(72) Inventors: Elly Bak, Rijsenhout (NL); Nicolaas D.M. Steur, Oude Niedorp (NL)

(21) Appl. No.: 15/999,224

(22) Filed: Aug. 16, 2018

(51) Int. Cl. A01H 6/00

(52) U.S. Cl. PLT/263.1

(57) ABSTRACT

A new and distinct Medinilla cultivar named ‘ROYAL TWIST’ is disclosed, characterized by an erect and sturdy plant habit. Glossy, green foliage is approximately 16 cm in length. Pendulous panicles are composed of flowers colored near Red-Purple 64C. The new variety is a Medinilla, normally produced as an ornamental plant.

Latin name of the genus and species: Medinilla magnifica.

Variety denomination: ‘ROYAL TWIST’.

### BACKGROUND OF THE INVENTION

The present invention relates to a new and distinct hybrid cultivar of Medinilla plant, botanically known as Medinilla sp., hereinafter referred to by the cultivar name ‘ROYAL TWIST’. The genus of Medinilla belongs to the Melastomataceae family.

The new Medinilla ‘ROYAL TWIST’ is a product of a planned breeding program conducted by the inventors, Elly Black and Nico D. M. Steur, in Assendelft, The Netherlands. The seed parent is the Medinilla selection designated ‘82347005’ (unpatented). The pollen parent is the Medinilla selection designated ‘82341131’ (unpatented). The objective of the breeding program was to produce a new Medinilla variety with a good growing habit and an attractive inflorescence. The crossing was made in 2008.

‘ROYAL TWIST’ was discovered and selected by the inventors in 2010, as a single flowering plant, at a research greenhouse in Assendelft, The Netherlands.

Asexual reproduction of the new Medinilla hybrid ‘ROYAL TWIST’ by vegetative means by terminal shoots was first performed in 2013 Assenfeldt, the Netherlands. Subsequent propagation has demonstrated that the combination of characteristics as herein disclosed for the new cultivar are firmly fixed and retained through successive generations of asexual reproduction. The new cultivar reproduces true-to-type.

### BRIEF DESCRIPTION OF THE INVENTION

The following traits have been repeatedly observed and are determined to be the unique characteristics of ‘ROYAL TWIST’ which in combination distinguish this Medinilla hybrid as a new and distinct cultivar:

1. Solid growth habit; plants are sturdy and not leggy.
2. Erect plant, measuring about 35 cm in height (above the pot when first flowering);
3. Glossy green colored leaves, measuring about 16 cm in length.
4. Pendulous panicles with red-purple (RHS 64C) colored flowers.

### COMMERCIAL COMPARISON

Of the commercial cultivars known to the present inventors, the most similar in comparison to the new Medinilla ‘ROYAL TWIST’ is the Medinilla ‘DOLCE VITA’, unpatented. Plants of the new Medinilla ‘ROYAL TWIST’ differ from plants of Medinilla ‘DOLCE VITA’ in the following ways:

1. Plants of ‘ROYAL TWIST’ are larger than ‘DOLCE VITA’.
2. Plants of ‘ROYAL TWIST’ are taller than ‘DOLCE VITA’.

### BRIEF DESCRIPTION OF THE PHOTOGRAPHS

The accompanying photographs illustrate the overall appearance of the new Medinilla ‘ROYAL TWIST’ showing the colors as true as is reasonably possible with colored reproductions of this type. Colors in the photographs may differ slightly from the color values cited in the detailed botanical description which accurately describes the color of ‘ROYAL TWIST’.

FIG. 1. shows a side view perspective of a typical potted flowering plant of ‘ROYAL TWIST’ at about 50 weeks from potting.

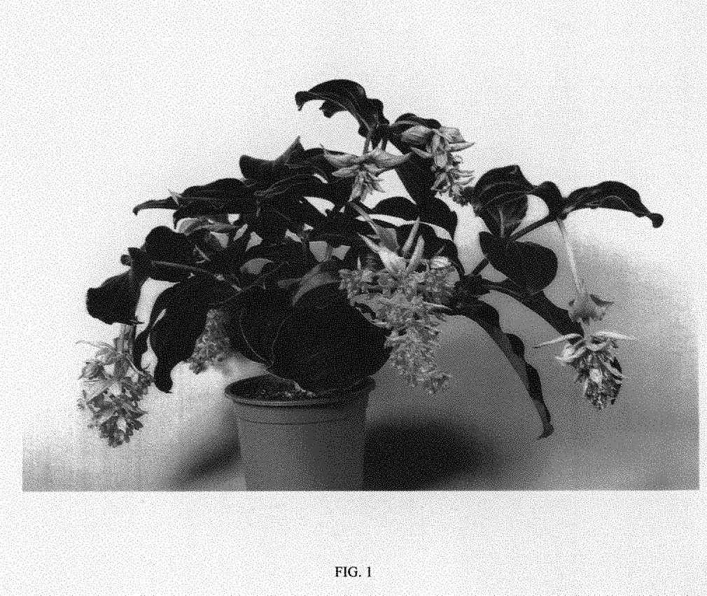

FIG. 2. shows a side view perspective of a typical inflorescence of ‘ROYAL TWIST’ at about 50 weeks from potting.

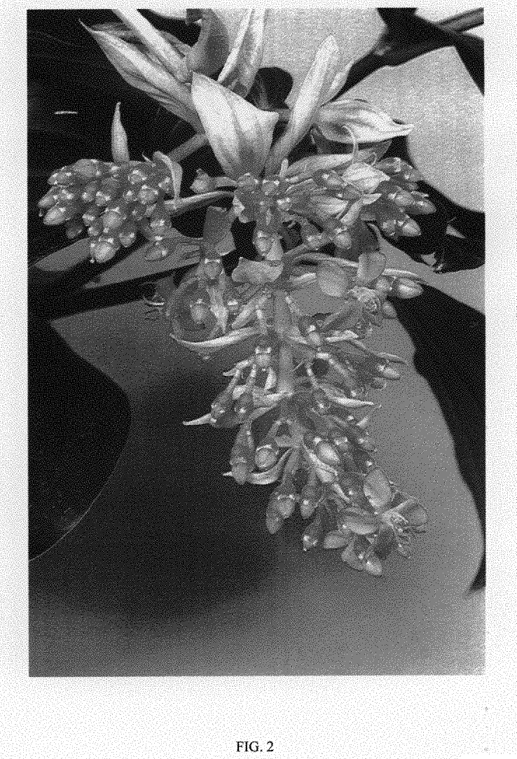

### DETAILED BOTANICAL DESCRIPTION

‘ROYAL TWIST’ has not been observed under all possible environmental conditions. The phenotype of the new cultivar may vary with variations in environment such as temperature, light intensity, day length, fertilizers and humidity without any change in the genotype of the plant.

The aforementioned photograph, together with the following observations, measurements and values describe the new Medinilla ‘ROYAL TWIST’ as grown in a greenhouse in Assendelft, the Netherlands, under conditions which closely approximate those generally used in a commercial practice.

Color references are made to The Royal Horticultural Society Colour Chart (R.H.S.), 2001Edition, except where general colors of ordinary significance are used. Color values were taken under daylight conditions in a greenhouse in Assendelft, The Netherlands. The age of the plants of ‘ROYAL TWIST’ described is about 50 weeks from potting.
Botanical classification: Medinilla magnifica ‘ROYAL TWIST’.
Parentage:
   Female parent.—Medinilla selection designated ‘82347005’ (unpatented).
   Male parent.—Medinilla selection designated ‘82341131’ (unpatented).
Propagation: Cuttings.
Plant:
   General appearance and form.—Height: About 35 cm. Diameter: About 60 cm. Shape: Outwardly erect, overall elliptic. Growth Habit: Tender indoor flowering ornamental. Plant Vigor: Good. Flowering Season: From April until October. Cold Tolerance: Frost tender, temperatures below 5° C. may damage plants. Fragrance: None.
Quantity of primary lateral branches: Average: 2.
Characteristics of primary lateral branches:
   Diameter.—About 15 mm.
   Shape.—Quandrangular, winged.
   Width of wings.—About 3 mm.
   Color.—Green; near RHS 137B with lignified parts near Grey-Brown 199B.
Foliage:
   Arrangement.—Opposite.
   Overall shape.—Elliptical.
   Quantity.—About 44.
   Attachment.—Sessile.
   Average overall length.—16 cm.
   Average overall width.—11 to 13 cm.
   Apex.—Cuspidate.
   Base.—Acute.
   Margin.—Strongly undulate.
   Texture.—Smooth, upper and lower surfaces.
   Appearance.—Glossy upper surface, matte lower surface.
   Color.—Color may vary depending on growing conditions. Upper Surface: Glossy Greyed-green, near RHS 189A. Lower Surface: Yellow-Green, near RHS 146B.
   Venation.—Type: Sunken, below surface of leaf. Pattern: Parallel. Color upper side: Yellow-green; closest to RHS 145C. Color under side: Green; closest to RHS 138B.
Inflorescence:
   Shape.—Pendulous panicles.
   Amount.—About 7, depending on size of plant.
   Length.—Average: 25 cm.
   Width.—Average: 14 cm.
   Color.—Red-Purple, closest to RHS 64C.
Corolla:
   Number.—About 50-200 per panicle.
Petals:
   Quantity.—4-5 per flower.
   Length.—Average: 13 mm.
   Width.—Average: 11 mm.
   Overall shape.—Obovate, asymmetrical.
   Apex.—Obtuse.
   Color.—Upper surface: Red-purple closest to RHS 74C. Lower surface: Red-purple closest to RHS 74C.
Sepals:
   Arrangement.—Growth together, enclosing the ovary.
   Color.—Near Red-Purple RHS 67A, stippled with White 155D.
Bracts:
   Arrangement.—Verticillate.
   Quantity.—4 per crown.
   Length.—Average 2 to 8 cm.
   Width.—Average 2 to 3.5 cm. Bracts become smaller closer to the top.
   Shape.—Elliptical.
   Color.—Green with Red-Purple, near RHS 143A and 57D.
Bud:
   Length.—Average: 10 mm.
   Diameter.—Average: 5 mm.
   Color.—Red-purple, near RHS 57C.
Peduncles:
   Length.—Average 18 cm.
   Diameter.—Average 7 mm.
   Color.—Green RHS 144B.
Pedicels:
   Length.—About 21 mm.
   Diameter.—Average: 2 mm.
   Color.—Closest to Red-purple RHS 57D.
Reproductive organs:
Stamens:
   Number.—Average 8 per flower.
   Length.—7 mm.
   Diameter.—1 mm.
   Color.—Yellow, with a white base (too small to qualify R.H.S. value).
Anthers:
   Length.—8 mm.
   Color.—Violet, closest to RHS 87A.
   Pollen.—Not visible.
Pistil:
   Number.—1 per flower.
   Length.—16 mm.
   Style.—Length: 12 mm. Color: White, closest to RHS 155D.
   Stigma.—Shape: Punctiform. Width: <1 mm. Color: White, too small to qualify R.H.S. value.
   Ovary.—Shape: Obovoid. Position: Inferior. Length: 4 mm. Diameter: 4.5 mm. Color: White; closest to RHS 155D.
Other characteristics:
Seeds and fruits: No seeds or fruits detected to date.
Disease/pest resistance: Neither resistance nor susceptibility to normal diseases and pests of Medinilla observed to date.
Temperature tolerance: Temperatures below 5° C. may damage plants. Upper temperature range unknown, tolerates at least 38° C.

What is claimed is:

1. A new and distinct cultivar of Medinilla plant named ‘ROYAL TWIST’ as herein illustrated and described.
